SEQUENCE LISTING (44 sequences)

SEQ ID NO: 1 - DNA, 46 bp, Artificial Sequence
  PCR primer
tcgactagtcctcccgggatcctcaggcctcgtgatacgcctattt

SEQ ID NO: 2 - DNA, 46 bp, Artificial Sequence
  PCR primer
tcccgggaggactagtcgacgttatcggcgagcggtatcagctcac

SEQ ID NO: 3 - DNA, 38 bp, Artificial Sequence
  Oligonucleotide with multiple cloning sites
gtcgacagctgcggaattctgctagcggatccagatct

SEQ ID NO: 4 - DNA, 23 bp, Artificial Sequence
  PCR primer
gacttgtcgggacggccctatgc

SEQ ID NO: 5 - DNA, 33 bp, Schizosaccharomyces pombe
ggcggcgatatcgaaagagctggctcgtttgac

SEQ ID NO: 6 - DNA, 33 bp, Schizosaccharomyces pombe
ccgagagacagctgcaacagcacaatgtatttg

SEQ ID NO: 7 - DNA, 27 bp, Schizosaccharomyces pombe
gctgttgcagctgtctctcggaactta

SEQ ID NO: 8 - DNA, 39 bp, Schizosaccharomyces pombe
gctatacgatatccggatccaactaaactaacttccaaa

SEQ ID NO: 9 - DNA, 20 bp, Schizosaccharomyces pombe
gctccgagttaattcgtggt

SEQ ID NO: 10 - DNA, 31 bp, Schizosaccharomyces pombe
ggcgggcagctgatgacttaacgcgaaacga

SEQ ID NO: 11 - DNA, 32 bp, Schizosaccharomyces pombe
atctgtgacccgggtttgcactcttcaaacat

SEQ ID NO: 12 - DNA, 30 bp, Schizosaccharomyces pombe
tgcaaacccgggtcacagatatgggtctat

SEQ ID NO: 13 - DNA, 39 bp, Schizosaccharomyces pombe
gctatacgatatccggatccacgcgaaaaagaatgcaag

SEQ ID NO: 14 - DNA, 20 bp, Schizosaccharomyces pombe
caaacatgtgggcctatgca

SEQ ID NO: 15 - DNA, 31 bp, Schizosaccharomyces pombe
ggaggcctcgagtctctttaactacccaatg

SEQ ID NO: 16 - DNA, 32 bp, Schizosaccharomyces pombe
tattacggatatccaaaggttaatttgaacat

SEQ ID NO: 17 - DNA, 28 bp, Schizosaccharomyces pombe
aacctttggatatccgtaataagtcgag

SEQ ID NO: 18 - DNA, 31 bp, Schizosaccharomyces pombe
ctcggagcagctgaacaaagtgaaacgtctc

SEQ ID NO: 19 - DNA, 32 bp, Schizosaccharomyces pombe
gagggcacttcctctgtaaagatccgtcatgc

SEQ ID NO: 20 - DNA, 33 bp, Schizosaccharomyces pombe
ggcggagatatccatatttcacatcctcatttt

SEQ ID NO: 21 - DNA, 31 bp, Schizosaccharomyces pombe
tcctcatcagctgtgattcttaatatgcagc

SEQ ID NO: 22 - DNA, 28 bp, Schizosaccharomyces pombe
aagaatcacagctgatgaggaggttgtc

SEQ ID NO: 23 - DNA, 39 bp, Schizosaccharomyces pombe
gtatgctatacgatggatccagcatagtaacttaacgcc

SEQ ID NO: 24 - DNA, 29 bp, Schizosaccharomyces pombe
ggaggagatatcgttgtccctttctttgc

SEQ ID NO: 25 - DNA, 29 bp, Schizosaccharomyces pombe
ggcggagatatctgcttcctcgagcatca

SEQ ID NO: 26 - DNA, 31 bp, Schizosaccharomyces pombe
taattgtcccgggcaaaacactaatattcgc

SEQ ID NO: 27 - DNA, 30 bp, Schizosaccharomyces pombe
tgttttgcccgggacaattaggaatttccc

SEQ ID NO: 28 - DNA, 40 bp, Schizosaccharomyces pombe
gctatacgatatccggatccaaaccattccaggctagatt

SEQ ID NO: 29 - DNA, 20 bp, Schizosaccharomyces pombe
ggcattcaattacagaatcc

SEQ ID NO: 30 - DNA, 29 bp, Schizosaccharomyces pombe
ggaggagtcgacctaagctatttatttgg

SEQ ID NO: 31 - DNA, 30 bp, Schizosaccharomyces pombe
aaacaatcagctgctgtggaatcaaagagt

SEQ ID NO: 32 - DNA, 31 bp, Schizosaccharomyces pombe
tccacagcagctgattgtttatactaatatc

SEQ ID NO: 33 - DNA, 29 bp, Schizosaccharomyces pombe
ggaggaagatctcacaagtgattgtgtgg

SEQ ID NO: 34 - DNA, 30 bp, Schizosaccharomyces pombe
ggaggacagctgtatgaaaagcagtgtttg

SEQ ID NO: 35 - DNA, 31 bp, Schizosaccharomyces pombe
ggaggactcgagctgtacataatattcccgc

SEQ ID NO: 36 - DNA, 31 bp, Schizosaccharomyces pombe
tatcaaccagctgtgatcgcattcatagccg

SEQ ID NO: 37 - DNA, 30 bp, Schizosaccharomyces pombe
tgcgatcacagctggttgatactcgaaagg

SEQ ID NO: 38 - DNA, 30 bp, Schizosaccharomyces pombe
ggaggagatatcatttaacttcatgcggcg

SEQ ID NO: 39 - DNA, 18 bp, Schizosaccharomyces pombe
cacctttaatagacccct

SEQ ID NO: 40 - DNA, 30 bp, Schizosaccharomyces pombe
ccgctcgagtcggcaagtttatgcaacttg

SEQ ID NO: 41 - DNA, 38 bp, Schizosaccharomyces pombe
cctacgttcatttgatgctgtcatggatatctcattgc

SEQ ID NO: 42 - DNA, 39 bp, Schizosaccharomyces pombe
gatatccatgacagcatcaaatgaacgtaggattagggg

SEQ ID NO: 43 - DNA, 30 bp, Schizosaccharomyces pombe
ggaggacagctggttatggtattcagcatg

SEQ ID NO: 44 - DNA, 17 bp, Schizosaccharomyces pombe
attctgctaaaacacct

(19) United States
(12) Patent Application Publication
(10) Pub. No.: US 2013/0053552 A1
(43) Pub. Date: Feb. 28, 2013

(54) FRAGMENT SWITCH: A REVERSE GENETIC APPROACH

(71) Applicants: Xie Tang, Singapore (SG); Mohan Balasubramanian, Singapore (SG)

(72) Inventors: Xie Tang, Singapore (SG); Mohan Balasubramanian, Singapore (SG)

(73) Assignee: TEMASEK LIFE SCIENCES LABORATORY LIMITED, Singapore (SG)

(21) Appl. No.: 13/643,876

(22) PCT Filed: Mar. 24, 2011
(86) PCT No.: PCT/SG2011/000116

§ 371 Date: Oct. 26, 2012

Related U.S. Application Data
(60) Provisional application No. 61/329,888, filed on Apr. 30, 2010.

(51) Int. Cl. C12N 15/65; C12N 15/11

(52) U.S. Cl. 536/23.1; 435/471

(57) ABSTRACT

The present invention relates to the field of reverse genetics. More particularly, the present invention relates to a novel reverse genetic approach termed “fragment switch” which is used to generate an allelic series in genes of interest which are useful for functional analysis.

### CROSS-REFERENCE TO RELATED APPLICATION

The present application is related to and claims priority to U.S. provisional patent application Ser. No. 61/329,888 filed on 30 Apr. 2010.

### SEQUENCE SUBMISSION

The present application is being filed along with a Sequence Listing in electronic format. The Sequence Listing is entitled 2577-202PCT_ST25.txt, was created on 11 Mar. 2011 and is 7.32 kb in size. The information in the electronic format of the Sequence Listing is part of the present application and is incorporated herein by reference in its entirety.

### BACKGROUND OF THE INVENTION

The present invention relates to the field of reverse genetics. More particularly, the present invention relates to a novel reverse genetic approach termed “fragment switch” which is used to generate an allelic series in genes of interest which are useful for functional analysis.

The publications and other materials used herein to illuminate the background of the invention or provide additional details respecting the practice, are incorporated by reference, and for convenience are respectively grouped in the Bibliography.

Genetic analysis using mutant strains have played important roles in the identification and functional analysis of genes. The phenotype-based forward genetic screens have contributed a lot to discovering genes involved in biosynthesis, cell cycle, and signaling pathways etc. In order to dissect multiple functions of some genes and the underlying molecular mechanisms, an allelic series becomes desirable. With the genomes sequenced, reverse genetic screens have become prevalent tools for generating mutant alleles, especially for genes that have not been identified by forward genetic screens.

There are several reverse genetic approaches for fission yeast, such as plasmid shuffling (Liang and Frosburg, 2001), degron system (Kanemaki et al., 2003), chromosomal integration and marker switch (MacIver et al., 2003). All these techniques have their own limitations. Plasmid shuffling is time consuming. Degron system creates only one loss of function temperature sensitive allele, and does not work for all genes because it requires an additional peptide to be tagged. Both chromosomal integration and marker switch use polymerase chain reaction (PCR) to amplify a long fragment that consists of a flanking sequence, a selective marker gene and the target gene (FIG. 1a). The long selective marker gene not only leads to less PCR product, but also has high chance to get unexpected mutations by mutagenic PCR, leading to inefficient swapping mutation into the target gene.

Therefore, a simpler and more efficient approach is desirable for reverse genetics.

### SUMMARY OF THE INVENTION

The present invention relates to the field of reverse genetics. More particularly, the present invention relates to a novel reverse genetic approach termed “fragment switch” which is used to generate an allelic series in genes of interest which are useful for functional analysis.

In one aspect, the present invention provides a method for generating an allelic series in a gene of interest. In one embodiment, a method for generating a mutant allele of a gene of interest comprises integrating a first nucleic acid fragment adjacent to a gene of interest in a first nucleic acid in a host organism's DNA, wherein the first nucleic acid fragment comprises (i) a gene encoding a first selectable marker having a carboxy terminus truncation or an amino terminus truncation and (ii) a gene encoding a second selectable marker. The method also comprises preparing a second nucleic acid fragment comprising (i) the gene of interest having one or more mutations and (ii) a fragment of the first selectable marker gene, wherein the fragment of the first selectable marker gene encodes the carboxy terminus or amino terminus of the first selectable marker. The method further comprises recombining the second nucleic acid fragment with the first nucleic acid under pressure of selection for the first selectable marker to produce a second nucleic acid comprising (i) the gene of interest having the one or more mutations, (ii) a gene encoding the first selectable marker and (iii) a gene encoding the second selectable marker.

In one embodiment, the nucleic acid fragment encoding the first selectable marker having a carboxy terminus truncation or an amino terminus truncation is integrated next to the gene of interest. In one embodiment, the truncation is a carboxy terminus truncation and the 3′ end of the gene encoding the first selectable marker is positioned on the 3′ side of the gene of interest. In another embodiment, the truncation is a carboxy terminus truncation and the 3′ end of the gene encoding the first selectable marker is positioned on the 5′ side of the gene of interest. In an additional embodiment, the truncation is an amino terminus truncation and the 5′ end of the gene encoding the first selectable marker is positioned on the 3′ side of the gene of interest. In a further embodiment, the truncation is an amino terminus truncation and the 5′ end of the gene encoding the first selectable marker is positioned on the 5′ side of the gene of interest.

In one embodiment, the fragment of the first selectable marker gene is located next to the gene of interest having the one or more mutations. In another embodiment, the carboxy terminus of the first selectable marker in the second fragment complements the first selectable marker having the carboxy terminus truncation or the amino terminus of the first selectable marker in the second fragment complements the first selectable marker having the amino terminus truncation. In one embodiment, the fragment of the first selectable marker gene encodes the carboxy terminus of the first selectable marker and the 3′ end of the fragment of the first selectable marker gene is positioned on the 3′ side of the gene of interest having the one or more mutations. In another embodiment, the fragment of the first selectable marker gene encodes the carboxy terminus of the first selectable marker and the 3′ end of the fragment of the first selectable marker gene is positioned on the 5′ side of the gene of interest having the one or more mutations. In an additional embodiment, the fragment of the first selectable marker gene encodes the amino terminus of the first selectable marker and the 5′ end of the fragment of the first selectable marker gene is positioned on the 3′ side of the gene of interest having the one or more mutations. In a further embodiment, the fragment of the first selectable marker gene encodes the amino terminus of the first selectable marker and the 5′ end of the fragment of the first selectable marker gene is positioned on the 5′ side of the gene of interest having the one or more mutations. In one embodiment, the second fragment can be produced by mutagenic PCR. In a further embodiment, the recombination is homologous recombination in a host organism.

In a second aspect, the present invention provides an isolated nucleic acid that comprises a gene encoding a first selectable marker having a carboxy terminus truncation or an amino terminus truncation and a gene encoding a second selectable marker. In one embodiment, at least the portion of the isolated nucleic acid containing the first selectable marker gene and the second selectable marker gene is capable of integrating into a host organism's DNA. In another embodiment, the second marker gene is positioned on the 5′ or 3′ side of the first marker gene depending on which end of the first marker gene integrates next to the gene of interest.

In a third aspect, the present invention provides a nucleic acid that comprises a gene of interest, a gene encoding a first selectable marker having a carboxy terminus truncation or an amino terminus truncation and a gene encoding a second selectable marker. In one embodiment, the gene encoding the first selectable marker having a carboxy terminus truncation is integrated next to the gene of interest as described herein. In another embodiment, the gene encoding the first selectable marker having an amino terminus truncation is integrated next to the gene of interest as described herein.

In a fourth aspect, the present invention provides an isolated nucleic acid that comprises a gene of interest having one or more mutations and a fragment of the first selectable marker gene which encodes the carboxy terminus or amino terminus of the first selectable marker. In one embodiment, the fragment of the first selectable marker gene is located next to the gene of interest having the one or more mutations as described herein. In another embodiment, at least the portion of the isolated nucleic acid containing the gene of interest having one or more mutations and the fragment of the first selectable marker gene is capable of integrating into a host organism's DNA.

In a fifth aspect, the present invention provides a nucleic acid that comprises a gene of interest having one or more mutations, a gene encoding a first selectable marker and a gene encoding a second selectable marker.

### BRIEF DESCRIPTION OF THE FIGURES

FIGS. 1a and 1b show marker switch and design of fragment switch. FIG. 1a: Marker switch. Selection marker 1 (sm1⁺) is inserted adjacent to gene of interest (goi⁺) through homologous recombination. Mutations (asterisk) generated by mutagenic PCR are delivered into chromosome together with the selection for selection marker 2 (sm2⁺), which replaces sm1⁺. FIG. 1b: A partial fragment (sm1^{Δc}) of sm1⁺ with a carboxyl terminus truncation is inserted adjacent to goi⁺ together with the selection for sm2⁺. Mutations are delivered into chromosome under the selection for sm1⁺, which is complemented by the recombination between sm1^{Δc} and sm1^{c}, a fragment of the carboxyl terminus of sm1⁺.

FIGS. 2a-2c show the two steps of fragment switch. FIG. 2a: Step one: plasmid-based strategy is used to insert his5^{Δc} adjacent to goi⁺. One upstream and one downstream fragments of the insertion locus, which have an overlap about 25 nucleotides and a unique restriction site (double arrows), are amplified, mixed, and then amplified again to generate a fusion fragment. This fusion fragment is cloned into plasmid pH5ΔcU4+. The resulting plasmid is restricted (scissor symbol) and transformed into a strain with the endogenous his5⁺ and ura4⁺ deleted, generating goi⁺-his5^{Δc}. FIG. 2b: Step two: mutations are delivered to goi⁺ together with the complementation of his5⁺. The whole goi⁺ is cloned into plasmid pH5c. Mutagenic PCR used to produce the fusion fragment goi^{m}-his5^{c}, which is subsequently transformed and recombined with goi⁺-his5^{Δc}. FIG. 2c: Domain-specific mutagenesis: mutagenic PCR is applied to a specific region and high fidelity PCR to the downstream part, and a second round of high fidelity PCR again to generate a fusion fragment in which most mutations are in the this specific region.

FIG. 3 shows mutants generated with fragment switch. Seven genes were picked to test the fragment switch approach. All mutants except ppk37-24 were temperature sensitive in rich medium YES (die at 36° C. but survive at 24° C.). Mutants were cultured in YES over night at 24° C., and then shifted to 36° C. for 4 hours. Cells were fixed with formaldehyde and stained with 4′,6-diamidino-2-phenylindole (DAPI) (DNA) and aniline blue (septum). The his5^{Δc} was inserted in the 3′-UTR region of the genes except myo2, in which it was inserted in both 5′- and 3′-UTR separately in two strains.

### DETAILED DESCRIPTION OF THE INVENTION

Unless defined otherwise, all technical and scientific terms used herein have the same meaning as is commonly understood by one of skill in the art to which the invention belongs.

The present invention relates to the field of reverse genetics. More particularly, the present invention relates to a novel reverse genetic approach termed “fragment switch” which is used to generate an allelic series in genes of interest which are useful for functional analysis.

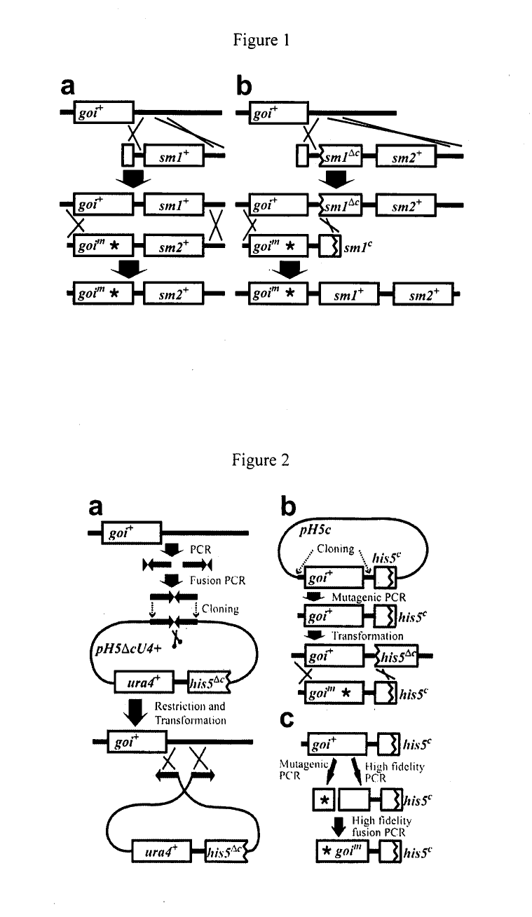

Mutagenesis by a reverse genetic approach requires optimal mutation frequency, efficient targeting, and selection pressure. Those could be fulfilled by mutagenic PCR (Vallette et al., 1989), homologous recombination and an efficient selective marker. As shown in FIG. 1a, marker switch approach consists of two steps: insertion of a first selectable marker (sm1⁺) next to a gene of interest (goi⁺) and incorporation of mutations into goi⁺ together with the replacement of sm1⁺ by a second selectable marker sm2⁺. Actually, chromosome integration only uses the first step to deliver mutation into the gene of interest, but marker switch has an advantage because it indirectly targets mutation incorporation into goi⁺ by excluding random integrations in other loci.

To simplify marker switch, it was combined with a targeted integration approach described previously (Keeney and Boeke, 1994). Two simple steps were designed to fulfill this approach (FIG. 1b). First, a fragment (sm1^{Δc}) of sm1⁺ with a carboxyl terminus truncation is integrated adjacent to goi⁺ together with another selection marker (sm2⁺). Second, mutagenic PCR is used to produce a fusion fragment between goi and the carboxy terminus of sm1⁺, goi^{m}-sm1^{c}, which harbors a mutation in goi and complements sm1^{Δc}. After this fusion fragment is transformed, mutations are delivered into goi through homologous recombination precisely between this fusion fragment and goi⁺-sm1^{Δc} under the pressure of selection for sm1⁺. This eliminates the need to further confirm the swapping as done by marker switch. Since only a partial fragment of the selective marker instead of a full length is used for integration by homologous recombination, this approach is called “fragment switch” herein.

In one aspect, the present invention provides a method for generating an allelic series in a gene of interest. In one embodiment, a method for generating a mutant allele of a gene of interest comprises integrating a first nucleic acid fragment adjacent to a gene of interest in a first nucleic acid in a host organism's DNA, wherein the first nucleic acid fragment comprises (i) a gene encoding a first selectable marker having a carboxy terminus truncation or an amino terminus truncation and (ii) a gene encoding a second selectable marker. The method also comprises preparing a second nucleic acid fragment comprising (i) the gene of interest having one or more mutations and (ii) a fragment of the first selectable marker gene, wherein the fragment of the first selectable marker gene encodes the carboxy terminus or amino terminus of the first selectable marker. The method further comprises recombining the second nucleic acid fragment with the first nucleic acid under pressure of selection for the first selectable marker to produce a second nucleic acid comprising (i) the gene of interest having the one or more mutations, (ii) a gene encoding the first selectable marker and (iii) a gene encoding the second selectable marker.

In one embodiment, the nucleic acid fragment encoding the first selectable marker having a carboxy terminus truncation or an amino terminus truncation is integrated next to the gene of interest. In one embodiment, the truncation is a carboxy terminus truncation and the 3’ end of the gene encoding the first selectable marker is positioned on the 3′ side of the gene of interest. In another embodiment, the truncation is a carboxy terminus truncation and the 3′ end of the gene encoding the first selectable marker is positioned on the 5′ side of the gene of interest. In an additional embodiment, the truncation is an amino terminus truncation and the 5′ end of the gene encoding the first selectable marker is positioned on the 3′ side of the gene of interest. In a further embodiment, the truncation is an amino terminus truncation and the 5′ end of the gene encoding the first selectable marker is positioned on the 5′ side of the gene of interest.

In one embodiment, the fragment of the first selectable marker gene is located next to the gene of interest having the one or more mutations. In another embodiment, the carboxy terminus of the first selectable marker in the second fragment complements the first selectable marker having the carboxy terminus truncation or the amino terminus of the first selectable marker in the second fragment complements the first selectable marker having the amino terminus truncation. In one embodiment, the fragment of the first selectable marker gene encodes the carboxy terminus of the first selectable marker and the 3′ end of the fragment of the first selectable marker gene is positioned on the 3′ side of the gene of interest having the one or more mutations. In another embodiment, the fragment of the first selectable marker gene encodes the carboxy terminus of the first selectable marker and the 3′ end of the fragment of the first selectable marker gene is positioned on the 5′ side of the gene of interest having the one or more mutations. In an additional embodiment, the fragment of the first selectable marker gene encodes the amino terminus of the first selectable marker and the 5′ end of the fragment of the first selectable marker gene is positioned on the 3′ side of the gene of interest having the one or more mutations. In a further embodiment, the fragment of the first selectable marker gene encodes the amino terminus of the first selectable marker and the 5′ end of the fragment of the first selectable marker gene is positioned on the 5′ side of the gene of interest having the one or more mutations. In one embodiment, the second fragment can be produced by mutagenic PCR. In a further embodiment, the recombination is homologous recombination in a host organism.

In addition to mutagenesis, fragment switch could be used for other reverse genetic manipulation such as precise carboxyl terminal tagging and even deletion. For precise carboxy terminal tagging, this method comprises preparing the second nucleis acid fragment comprising (i) the gene of interest with the stop codon deleted, (ii) a tagging fragment (such as gfp) fused with the gene of interest in frame, (iii) a fragment of the first selectable marker gene which encodes the carboxy terminus of the first selectable marker. For precise deletion, this method comprises preparing the second nucleis acid fragment comprising (i) a fragment upstream a gene of interest, (ii) a fragment of the first selectable marker gene which encodes the carboxy terminus of the first selectable marker. The method of the present invention can be used in any host organism in which homologous recombination can be performed. In addition to yeast that is illustrated herein (Schizosaccharomyces pombe), other organisms include, but are not limited to the bacterium Escherichia coli, the yeast Saccharomyces cerevisiae.

The gene of interest may be any gene for which it is desired to create an allelic series that is useful in an analysis of gene function by a reverse genetic approach. In the fact, this method is applicable to any gene for mutagenesis as long as the phenotype of the mutants is observable. Examples of genes include, but are not limited to three classes of genes in fission yeast: (i) genes encoding kinases (Bimbo et al., 2005), such as ppk37, ark1, cdc2, cdc7, cdk9, hsk1, orb6, plo1, ran1, shk1, sid1, sid2, ppk19, and genes encoding phosphatase, including cdc25, fcp1. (ii) genes encoding proteins essential for the functional cellular cytoskeleton, including act1, arp3, cdc12, rng2, myo2, rng3, adf1, nda3, nda2, alp1, alp21, alp6, alp4, pcp1. (iii) genes involving in signaling cascade, such as genes for septum initiating network, plo1, byr4, cdc16, spg1, cdc7, cdc11, sid4, sid1, sid2.

The mutation may be any mutation that leads to a loss of function or a gain of function. Thus, the mutation may be a nonsense mutation, a frameshift mutation, an insertion mutation, a deletion mutation or a missense mutation, each of which would lead to a loss of function or a gain of function.

Any selectable marker gene can be used in the present invention as long as it is compatible with and expressed in the host organism. Examples of selective markers in fission yeast include, but are not limited to, his5+and ura4. Examples of selectable markers in budding yeast include, but are not limited to, HIS3+ and URA3. Examples of selectable markers in Escherichia coli include, but are not limited to, bla, hyrR, and kanR. In one embodiment, the gene encoding the selectable marker includes the native promoter. In another embodiment, the gene encoding the selectable marker includes a heterologous promoter. Any heterologous promoter may be used as long as it functions in the host organism.

In a second aspect, the present invention provides an isolated nucleic acid that comprises a gene encoding a first selectable marker having a carboxy terminus truncation or an amino terminus truncation and a gene encoding a second selectable marker. In one embodiment, at least the portion of the isolated nucleic acid containing the first selectable marker gene and the second selectable marker gene is capable of integrating into a host organism's DNA. In another embodiment, the second marker gene is positioned on the 5′ or 3′ side of the first marker gene depending on which end of the first marker gene integrates next to the gene of interest. The selectable markers may be a selectable marker as described herein.

In a third aspect, the present invention provides a nucleic acid that comprises a gene of interest, a gene encoding a first selectable marker—having a carboxy terminus truncation or an amino terminus truncation and a gene encoding a second selectable marker. In one embodiment, the gene encoding the first selectable marker having a carboxy terminus truncation is integrated next to the gene of interest as described herein. In another embodiment, the gene encoding the first selectable marker having an amino terminus truncation is integrated next to the gene of interest as described herein. The gene of interest may be a gene as described herein. The selectable markers may be a selectable marker as described herein.

In a fourth aspect, the present invention provides an isolated nucleic acid that comprises a gene of interest having one or more mutations and a fragment of the first selectable marker gene which encodes the carboxy terminus or amino terminus of the first selectable marker. In one embodiment, the fragment of the first selectable marker gene is located next to the gene of interest having the one or more mutations as described herein. In another embodiment, at least the portion of the isolated nucleic acid containing the gene of interest having one or more mutations and the fragment of the first selectable marker gene is capable of integrating into a host organism's DNA. The gene of interest may be a gene as described herein. The selectable markers may be a selectable marker as described herein. The mutation may be a mutation as described herein.

In a fifth aspect, the present invention provides a nucleic acid that comprises a gene of interest having one or more mutations, a gene encoding a first selectable marker and a gene encoding a second selectable marker. The gene of interest may be a gene as described herein. The selectable markers may be a selectable marker as described herein. The mutation may be a mutation as described herein.

The practice of the present invention employs, unless otherwise indicated, conventional techniques of chemistry, molecular biology, microbiology, recombinant DNA, genetics, immunology, cell biology, cell culture and transgenic biology, which are within the skill of the art. See, e.g., Maniatis et al., 1982, Molecular Cloning (Cold Spring Harbor Laboratory Press, Cold Spring Harbor, N.Y.); Sambrook et al., 1989, Molecular Cloning, 2nd Ed. (Cold Spring Harbor Laboratory Press, Cold Spring Harbor, N.Y.); Sambrook and Russell, 2001, Molecular Cloning, 3rd Ed. (Cold Spring Harbor Laboratory Press, Cold Spring Harbor, N.Y.); Ausubel et al., 1992), Current Protocols in Molecular Biology (John Wiley & Sons, including periodic updates); Glover, 1985, DNA Cloning (IRL Press, Oxford); Russell, 1984, Molecular biology of plants: a laboratory course manual (Cold Spring Harbor Laboratory Press, Cold Spring Harbor, N.Y.); Anand, Techniques for the Analysis of Complex Genomes, (Academic Press, New York, 1992); Guthrie and Fink, Guide to Yeast Genetics and Molecular Biology (Academic Press, New York, 1991); Harlow and Lane, 1988, Antibodies, (Cold Spring Harbor Laboratory Press, Cold Spring Harbor, N.Y.); Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Riott, Essential Immunology, 6th Edition, Blackwell Scientific Publications, Oxford, 1988; Fire et al., RNA Interference Technology: From Basic Science to Drug Development, Cambridge University Press, Cambridge, 2005; Schepers, RNA Interference in Practice, Wiley-VCH, 2005; Engelke, RNA Interference (RNAi): The Nuts & Bolts of siRNA Technology, DNA Press, 2003; Gott, RNA Interference, Editing, and Modification: Methods and Protocols (Methods in Molecular Biology), Human Press, Totowa, N.J., 2004; Sohail, Gene Silencing by. RNA Interference: Technology and Application, CRC, 2004.

### EXAMPLES

The present invention is described by reference to the following Examples, which is offered by way of illustration and is not intended to limit the invention in any manner. Standard techniques well known in the art or the techniques specifically described below were utilized.

### Example 1

### Preparation of Plasmids for Fragment Switch in Yeast

Two plasmids, pH5ΔcU4+ (FIG. 2a) and pH5c (FIG. 2b), were constructed for the integration of sm1^{Δc} next to goi⁺ and generation of the fusion fragment, goi^{m}-sm1^{c}, respectively. Plasmid pN1 was constructed by PCR-based amplifying and circulizing a fragment using pUC18 (Sambrook and Russell, 2001, Molecular Cloning, 3rd Ed.) as the template and two overlapping primers (tcgactagtcctcccgggatcctcaggcctcgtgatacgcctattt (SEQ ID NO:1) and tcccgggaggactagtcgacgttatcggcgagcggtatcagctcac (SEQ ID NO:2)). The vector pH5ΔcU4+ was constructed by sequentially adding the functional selective maker ura4+and the nonfunctional selective marker his5^{Δc}, and a linker with multiple cloning sites (gtcgacagctgcggaattctgctagcggatccagatct (SEQ ID NO:3)) into pN1. The plasmid pH5c was constructed by sequentially adding the carboxy terminus of his5+ selective marker and the same linker in pH5ΔcU4+. The selection marker his5⁺ was chosen as sm1⁺ due to its effectiveness obtained during transformation (Tang et al., 2006) and its short coding sequence (651 base-pairs; GenBank Accession No. NM_{—}001021840). In his5^{Δc}, fifty base-pairs of the carboxyl terminus were removed, which made it nonfunctional. To insert his5^{Δc} adjacent to goi⁺, we used a plasmid-based integration strategy (Wang et al., 2004). As shown in FIG. 2a, PCR was used to generate one upstream and one downstream fragment of the insert locus separately. These two fragments overlapped 20˜30 base-pairs in their outward ends (the double head-to-head arrows), which carry a unique endonucuclease restriction site. These two fragments were mixed and used as the templates for the next round of fusion PCR. The fusion PCR product was cloned into the vector pH5ΔcU4+. The resulting plasmid was restricted, generating two ends that fully matched with the sequences on the chromosome. The linearized plasmid was transformed into the host organism (Schizosaccharomyces pombe) using the method as described (Okazaki, K. et al., 1990) and forced to undergo homologous recombination by the selection for ura4⁺, resulting in the integration of his5^{Δc} next to goi⁺. To introduce mutation into goi⁺, it was cloned into the vector pH5c to generate a fusion (goi⁺-his5^{c}) between goi⁺ and a 400-base-pair fragment containing a 3′-UTR and the carboxyl terminus part of his5⁺. The fusion fragment goi⁺-his5^{c} was used as the template to amplify goi^{m}-his5^{c} by mutagenic PCR using one primer (MOH3724his5c25u: gacttgtcgggacggccctatgc; SEQ ID NO:4) specific to the 5′ end of goi+ and one specific to his5c. After goi^{m}-his5^{c} was transformed and recombined with goi⁺-his5^{Δc}, mutations were delivered to goi upon the selection for his5⁺.

### EXAMPLE 2

### Preparation of Mutant Alleles of Genes of Interest

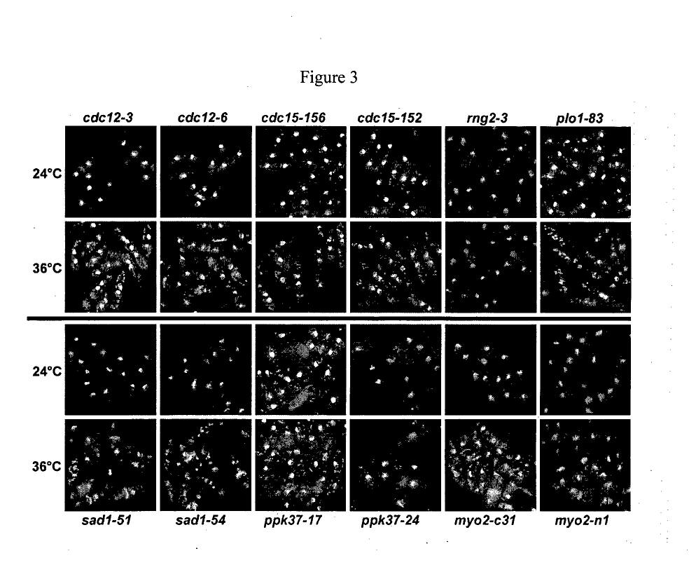

To test fragment switch, we performed proof-of-principle experiments to isolate mutant alleles for several genes of interest. We picked five genes essential for cytokinesis (Balasubramanian et al., 2004), cdc12, cdc15, rng2, plo1, myo2, one gene essential for spindle formation and function (Hagan and Yanagida, 1995), sad1, and one gene encoding a kinase with unknown function (Bimbó et al., 2005), ppk37 (FIG. 3). All the strains were grown in standard medium (Moreno et al., 1991) and transformation of Schizosaccharomyces pombe was done as described (Okazaki, K. et al., 1990). Details of genes of interest and the primers used are listed in Table 1.

TABLE 1
Genes of Interest and Primers
cdc12: SPAC1F5.04c, encoding Cdc12p, which is required for the assembly of the actin-
myosin ring for cytokinesis. Cdc12p nucleates actin filaments that grow rapidly from their
barbed ends in the presence of profilin.

| | | |
|---|---|---|
| Primers* | #1 | GGCGGCGATATCGAAAGAGCTGGCTCGTTTGAC (SEQ ID NO: 5) |
| | #2 | CCGAGAGACAGCTGCAACAGCACAATGTATTTG (SEQ ID NO: 6) |
| | #3 | GCTGTTGCAGCTGTCTCTCGGAACTTA (SEQ ID NO: 7) |
| | #4 | GCTATACGATATCCGGATCCAACTAAACTAACTTCCAAA (SEQ ID NO: 8) |
| | #5 | GCTCCGAGTTAATTCGTGGT (SEQ ID NO: 9) |

cdc15: SPAC20G8.05c, encoding Cdc15p, which is required for assembly and stabilization of
the actin-myosin ring for cytokinesis.

| | | |
|---|---|---|
| Primers* | #1 | GGCGGGCAGCTGATGACTTAACGCGAAACGA (SEQ ID NO: 10) |
| | #2 | ATCTGTGACCCGGGTTTGCACTCTTCAAACAT (SEQ ID NO: 11) |
| | #3 | TGCAAACCCGGGTCACAGATATGGGTCTAT (SEQ ID NO: 12) |
| | #4 | GCTATACGATATCCGGATCCACGCGAAAAAGAATGCAAG (SEQ ID NO: 13) |
| | #5 | CAAACATGTGGGCCTATGCA (SEQ ID NO: 14) |

rng2: SPAC4F8.13c, encoding Rng2p, which is required for assembly and contraction of the
actin-myosin ring for cytokinesis.

| | | |
|---|---|---|
| Primers* | #1 | GGAGGCCTCGAGTCTCTTTAACTACCCAATG (SEQ ID NO: 15) |
| | #2 | TATTACGGATATCCAAAGGTTAATTTGAACAT (SEQ ID NO: 16) |
| | #3 | AACCTTTGGATATCCGTAATAAGTCGAG (SEQ ID NO: 17) |
| | #4 | CTCGGAGCAGCTGAACAAAGTGAAACGTCTC (SEQ ID NO: 18) |
| | #5 | GAGGGCACTTCCTCTGTAAAGATCCGTCATGC (SEQ ID NO: 19) |

plo1: SPAC23C11.16, encoding Plo1p, which is required to form a bipolar spindle, the actin
ring and septum, and for septal material deposition.

| | | |
|---|---|---|
| Primers* | #1 | GGCGGAGATATCCATATTTCACATCCTCATTTT (SEQ ID NO: 20) |
| | #2 | TCCTCATCAGCTGTGATTCTTAATATGCAGC (SEQ ID NO: 21) |
| | #3 | AAGAATCACAGCTGATGAGGAGGTTGTC (SEQ ID NO: 22) |
| | #4 | GTATGCTATACGATGGATCCAGCATAGTAACTTAACGCC (SEQ ID NO: 23) |
| | #5 | GGAGGAGATATCGTTGTCCCTTTCTTTGC (SEQ ID NO: 24) |

sad1: SPBC12D12.01, encoding Sad1p, which is a component of the spindle pole body and is
involved in linking telomeres to the spindle pole body during meiotic prophase.

| | | |
|---|---|---|
| Primers* | #1 | GGCGGAGATATCTGCTTCCTCGAGCATCA (SEQ ID NO: 25) |
| | #2 | TAATTGTCCCGGGCAAAACACTAATATTCGC (SEQ ID NO: 26) |
| | #3 | TGTTTTGCCCGGGACAATTAGGAATTTCCC (SEQ ID NO: 27) |
| | #4 | GCTATACGATATCCGGATCCAAACCATTCCAGGCTAGATT (SEQ ID NO: 28) |
| | #5 | GGCATTCAATTACAGAATCC (SEQ ID NO: 29) |

ppk37: SPCC70.05c, encoding a kinase, which is essential for viability and which function is
not known.

| | | |
|---|---|---|
| Primers* | #1 | GGAGGAGTCGACCTAAGCTATTTATTTGG (SEQ ID NO: 30) |
| | #2 | AAACAATCAGCTGCTGTGGAATCAAAGAGT (SEQ ID NO: 31) |
| | #3 | TCCACAGCAGCTGATTGTTTATACTAATATC (SEQ ID NO: 32) |
| | #4 | GGAGGAAGATCTCACAAGTGATTGTGTGG (SEQ ID NO: 33) |
| | #5 | GGAGGACAGCTGTATGAAAAGCAGTGTTTG (SEQ ID NO: 34) |

myo2: SPCC645.05c, encoding Myo2p, which is involved in generating force for actin-myosin
ring constraction, and also binds to cdc4 and rlc1.

| | | |
|---|---|---|
| Primers | #1 | GGAGGACTCGAGCTGTACATAATATTCCCGC (SEQ ID NO: 35) |
| for amino- | #2 | TATCAACCAGCTGTGATCGCATTCATAGCCG (SEQ ID NO: 35) |
| terminus* | #3 | TGCGATCACAGCTGGTTGATACTCGAAAGG (SEQ ID NO: 37) |
| | #4 | GGAGGAGATATCATTTAACTTCATGCGGCG (SEQ ID NO: 38) |
| | #5 | GCGCACCTTTAATAGACCCCT (SEQ ID NO: 39) |
| Primers | #1 | CCGCTCGAGTCGGCAAGTTTATGCAACTTG (SEQ ID NO: 40) |
| for | #2 | CCTACGTTCATTTGATGCTGTCATGGATATCTCATTGC (SEQ ID NO: 41) |
| carboxy | #3 | GATATCCATGACAGCATCAAATGAACGTAGGATTAGGGG (SEQ ID NO: 42) |
| terminus* | #4 | GGAGGACAGCTGGTTATGGTATTCAGCATG (SEQ ID NO: 43) |
| | #5 | ATTCTGCTAAAACACCT (SEQ ID NO: 44) |

*Primer pair of #1 and #2, pair of #3 and #4, were used to amplify the outwards and inwards fragment of the gene of interest from the insertion site of his 5^{Δc}-ura4⁺. Then the fusion fragment of these two fragments was amplified using primer #1 and #4 and then cloned in pH5ΔcU4+. To construct fusion goi⁺-his5^{c}, primer #5 and #4 were used to amplify, which was then cloned in pH5c.

We obtained three rng2 mutants from less than one hundred his5⁺ transformants, one displayed temperature sensitive with multiple nuclei at 36° C. We did small-scale screenings for cdc12, sad1, and ppk37 using only two plates (150 mm diameter), and collected 11, 28, 30 mutants respectively. Two of each are,shown in FIG. 3. Both cdc12-3 and cdc12-6 died at 36° C.; cdc12-3 showed completely loss of septum formation, but cdc12-6 showed partial formation and disorganized septum. Both sad1 mutants showed unequal segregation of chromosomes, but sad1-54 showed multiple DNA foci in most of the cells at 36° C. Most of the ppk37 mutants showed slow growth in rich medium, arrested in different cell cycle stage like ppk37-17 at 36° C., and lysed on agar medium (data not shown). Therefore, ppk37 could be involved in membrane structure assembly. Six mutants like ppk37-24 showed double length as wild type and defects in septum formation. Most of plo1 mutants were elongated with multiple nuclei and defective septum formation except that plo1-83 only showed multiple nuclei and other four only showed defective in septum positioning (data not shown). Because myo2 has a long coding sequence (4581 base-pairs), his5^{Δc} was either inserted in the 3′UTR for mutagenesis in the carboxyl terminus of myo2, or upstream of promoter for mutagenesis in the amino terminus. Most of the myo2 mutants showed multiple nuclei and defective septum formation as in myo2-c31 and myo2-n1.

The method of the present invention as illustrated in these Examples is simple, consisting of only two steps. In the first step, the integration of his5^{Δc} next to goi⁺ is efficient because the insertion does not disrupt the function of goi⁺. In the second step, because only a short carboxyl terminus fragment his5^{Δc} is used to complement his5^{Δc}, homologous recombination has to undergo precisely between goi⁺-his5^{c} and goi⁺-his5^{Δc}, avoiding integration at any other loci. Compared to marker switch, fragment switch provides a much easier way to control the most important part: mutagenic PCR. This is because fragment switch only need about 200 base-pairs of the carboxyl terminus of the selection marker, which is hardly incorporated with mutations by PCR. We tested fragment switch on several genes, even on the carboxyl terminal and amino terminal regions of myo2. Also, mutations could be primarily introduced into a small domain using fusion PCR (FIG. 2c), which will be useful for domain functional analysis. In addition to mutagenesis, fragment switch could be used for other reverse genetic manipulation such as precise carboxyl terminal tagging and even deletion. It could be adapted to other model organisms in which homologous recombination can be performed.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited, to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. For example, if the range 10-15 is disclosed, then 11, 12, 13, and 14 are also disclosed. All methods described herein can be performed in any suitable order unless otherwise indicated herein or otherwise clearly contradicted by context. The use of any and all examples, or exemplary language. (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

It will be appreciated that the methods and compositions of the instant invention can be incorporated in the form of a variety of embodiments, only a few of which are disclosed herein. Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### BIBLIOGRAPHY

Balasubramanian, M. K. et al. (2004). Comparative analysis of cytokinesis in budding yeast, fission yeast and animal cells. Curr Biol 14: R806-818.

Bimbó, A. et al. (2005). Systematic deletion analysis of fission yeast protein kinases. Eukaryot Cell 4:799-813.

Hagan, I. and Yanagida, M. J. (1995). The product of the spindle formation gene sad1+ associates with the fission yeast spindle pole body and is essential for viability. J Cell Biol 129:1033-1047.

Kanemaki, M. et al. (2003). Functional proteomic identification of DNA replication proteins by induced proteolysis in vivo. Nature 423:720-724.

Keeney, J. B. and Boeke, J. D. (1994). Efficient targeted integration at leu1-32 and ura4-294 in Schizosaccharomyces pombe. Genetics 136:849-856.

Liang, D. T. and Forsburg, S. L. (2001). Characterization of Schizosaccharomyces pombe mcm7(+) and cdc23(+) (MCM10) and interactions with replication checkpoints. Genetics 159:471-486.

MacIver, F. H. et al. (2003). A ‘marker switch’ approach for targeted mutagenesis of genes in Schizosaccharomyces pombe. Yeast 20:587-594.

Moreno, S. et al. (1991). Molecular genetic analysis of fission yeast Schizosaccharomyces pombe. Methods Enzymol 194:7.95-823.

Okazaki, K. et al. (1990). High-frequency transformation method and library transducing vectors for cloning mammalian cDNAs by trans-complementation of Schizosaccharomyces pombe. Nucl. Acids Res 18:6485-6489.

Tang, X. et al. (2006). Bqt2p is essential for initiating telomere clustering upon pheromone sensing in fission yeast. J Cell Biol 173:845-851.

Vallette, F. et al. (1989). Construction of mutant and chimeric genes using the polymerase chain reaction. Nucleic Acids Res 17:723-733.

Wang, L. et al. (2004). Strategies for gene disruptions and plasmid constructions in fission yeast. Methods 33:199-205.

What is claimed is:

1. A method of generating an allelic mutant in a gene of interest comprising:
(a) integrating a first nucleic acid fragment adjacent to a gene of interest in a first nucleic acid, wherein the first nucleic acid fragment comprises (i) a gene encoding a first selectable marker having a carboxy terminus truncation or an amino terminus truncation and (ii) a gene encoding a second selectable marker;
(b) preparing a second nucleic acid fragment comprising (i) the gene of interest having one or more mutations and (ii) a fragment of the first selectable marker gene, wherein the fragment of the first selectable marker gene encodes the carboxy terminus or amino terminus of the first selectable marker, and wherein the fragment of the first selectable marker gene is positioned adjacent to the gene of interest having the one or more mutations; and
(c) recombining the second nucleic acid fragment with the first nucleic acid under pressure of selection for the first selectable marker to produce a second nucleic acid molecule comprising (i) the gene of interest having the one or more mutations, (ii) a gene encoding the first selectable marker and (iii) a gene encoding the second selectable marker,
whereby an allelic mutant in the gene of interest is generated.

2. The method of claim 1, wherein the first selectable marker gene having a carboxy terminus truncation or amino terminus truncation is integrated next to the gene of interest.

3. The method of claim 2, wherein the truncation is a carboxy terminus truncation and the 3′ end of the gene encoding the first selectable marker is positioned on the 3′ side of the gene of interest.

4. The method of claim 2, wherein the truncation is a carboxy terminus truncation and the 3′ end of the gene encoding the first selectable marker is positioned on the 5′ side of the gene of interest.

5. The method of claim 2, wherein the truncation is an amino terminus truncation and the 5′ end of the gene encoding the first selectable marker is positioned on the 3′ side of the gene of interest.

6. The method of claim 2, wherein the truncation is an amino terminus truncation and the 5′ end of the gene encoding the first selectable marker is positioned on the 5′ side of the gene of interest.

7. The method of claim 1, wherein the fragment of the first selectable marker gene encoding the carboxy terminus or amino terminus of the first selectable marker is located next to the gene of interest having the one or more mutations.

8. The method of claim 7, wherein the fragment of the first selectable marker gene encodes the carboxy terminus of the first selectable marker and the 3′ end of the fragment of the first selectable marker gene is positioned on the 3′ side of the gene of interest having the one or more mutations.

9. The method of claim 7, wherein the fragment of the first selectable marker gene encodes the carboxy terminus of the first selectable marker and the 3′ end of the fragment of the first selectable marker gene is positioned on the 5′ side of the gene of interest having the one or more mutations.

10. The method of claim 7, wherein the fragment of the first selectable marker gene encodes the amino terminus of the first selectable marker and the 5′ end of the fragment of the first selectable marker gene is positioned on the 3′ side of the gene of interest having the one or more mutations.

11. The method of claim 7, wherein the fragment of the first selectable marker gene encodes the amino terminus of the first selectable marker and the 5′ end of the fragment of the first selectable marker gene is positioned on the 5′ side of the gene of interest having the one or more mutations.

12. The method of claim 1, wherein the recombination is homologous recombination.

13. An isolated nucleic acid comprising a gene encoding a first selectable marker having a carboxy terminus truncation or an amino terminus truncation and a gene encoding a second selectable marker.

14. The isolated nucleic acid of claim 13, wherein at least the portion of the isolated nucleic acid containing the first selectable marker gene and the second selectable marker gene is capable of integrating into a host organism's DNA.

15. A nucleic acid comprising a gene of interest, a gene encoding a first selectable marker having a carboxy terminus truncation or an amino terminus truncation and a gene encoding a second selectable marker.

16. The nucleic acid of claim 15, wherein the truncation is a carboxy terminus truncation and the 3′ end of the gene encoding the first selectable marker is positioned on the 3′ side of the gene of interest.

17. The nucleic acid of claim 15, wherein the truncation is a carboxy terminus truncation and the 3′ end of the gene encoding the first selectable marker is positioned on the 5′ side of the gene of interest.

18. The nucleic acid of claim 15, wherein the truncation is an amino terminus truncation and the 5′ end of the gene encoding the first selectable marker is positioned on the 3′ side of the gene of interest.

19. The nucleic acid of claim 15, wherein the truncation is an amino terminus truncation and the 5′ end of the gene encoding the first selectable marker is positioned on the 5′ side of the gene of interest.

20. An isolated nucleic acid comprising a gene of interest having one or more mutations and a fragment of the first selectable marker gene which encodes the carboxy terminus or the amino terminus of the first selectable marker.

21. The isolated nucleic acid of claim 20, wherein the fragment of the first selectable marker gene encodes the carboxy terminus of the first selectable marker and the 3′ end of the fragment of the first selectable marker gene is positioned on the 3′ side of the gene of interest having the one or more mutations.

22. The isolated nucleic acid of claim 20, wherein the fragment of the first selectable marker gene encodes the carboxy terminus of the first selectable marker and the 3′ end of the fragment of the first selectable marker gene is positioned on the 5′ side of the gene of interest having the one or more mutations.

23. The isolated nucleic acid of claim 20, wherein the fragment of the first selectable marker gene encodes the amino terminus of the first selectable marker and the 5′ end of the fragment of the first selectable marker gene is positioned on the 3′ side of the gene of interest having the one or more mutations.

24. The isolated nucleic acid of claim 20, wherein the fragment of the first selectable marker gene encodes the amino terminus of the first selectable marker and the 5′ end of the fragment of the first selectable marker gene is positioned on the 5′ side of the gene of interest having the one or more mutations.

25. The isolated nucleic acid of claim 20, wherein at least the gene of interest having the one or more mutations and the fragment of the first selectable marker gene is capable of integrating into a host organism's DNA.

26. A nucleic acid that comprises a gene of interest having one or more mutations, a gene encoding a first selectable marker and a gene encoding a second selectable marker.
